(19) United States
(12) Patent Application Publication
(10) Pub. No.: US 2005/0260725 A1
(43) Pub. Date: Nov. 24, 2005

(54) Process for the preparation of trans-carveol

(76) Inventors: Wouter Adriaan Duetz, Zurich (CH); Bernard Witholt, Zurich (CH); Catherine Jourdat, Sainte Foy Les Lyon (FR)

Correspondence Address: HOFFMANN & BARON, LLP, 6900 JERICHO TURNPIKE, SYOSSET, NY, 11791, US

(21) Appl. No.: 10/416,125

(22) PCT Filed: Nov. 8, 2001
(86) PCT No.: PCT/EP01/13044

§ 371 Date: Sep. 5, 2003

(30) Foreign Application Priority Data
Nov. 10, 2000 (EP) 00124623.0

(51) Int. Cl. C12P 7/22

(52) U.S. Cl. 435/156

(57) ABSTRACT

A process for the production of trans-carveol which process comprises (a) incubading in the presence of D-limonene, a culture medium comprising microbial cells expressing an oxygenase capable of toluene and/or napththalene and (b) recovering the trans-carveol from the culture medium.

The present invention relates to a process for the preparation of monoterpenes, in particular trans-carveol using a microbial cell culture expressing an oxygenase capable of dioxygenation of toluene and/or naphthalene to the corresponding dihydrodiols.

D-limonene is the main constituent of orange and lemon peel oil which is a by-product of the orange juice industry in quantities of approximately 50 000 tons per year. Its low price makes it an attractive starting compound for industrially relevant fine-chemicals and flavour compounds with identical carbon-skeletons, such as carveol, carvone, and perillyl alcohol. The regiospecific introduction of carbonyl or hydroxy groups by chemical catalysis, however, is difficult because the electronic properties of the allylic methylene groups (carbon 1 and 2) and the allylic methyl groups (carbon 5 and 6) are rather similar. For this reason, enzymatic oxidation has been considered as early as the 1960's and numerous D-limonene transforming bacterial and plant cells have been reported on since.

Until now, the only biocatalysts showing an absolute regiospecificity were derived from plants. The P450 enzymes limonene 3-hydroxylase and limonene-6-hydroxylase (isolated from peppermint and spearmint microsomes) convert their natural substrate L-limonene and—at lower rates—D-limonene to isopiperitenol and carveol respectively. Another example is the oxidation of D-limonene in the 6 position to cis- and trans-carveol and carvone by Solanum aviculare and Dioscorea deltoidea. The specific activities of these plant enzymes, however, are insufficient for industrial applications.

Microbial strains found so far to be capable of bioconversion of D-limonene generally yielded a mixture of oxidation products. Recent examples are the conversion of D-Limonene to α-terpineol and 6-hydroxy-carveol by the honey fungus Arnmillareira mellae; to isopiperitenone, limonene-1,2 trans diol, cis-carveol and perillyl-alcohol, isopiperitenol, and α-terpineol by Aspergillus cellulosae; to carveol α-terpineol, perillyl alcohol and perillyl aldehyde by the D-limonene degrading strain Bacillus stearothermophilus BR388, and the same conversions by an Escherichia coli construct containing a plasmid with chromosomal inserts from this strain The most regiospecific microbial biocatalyst reported so far is the basidiomycete Pleurotus sapidus which converts D-limonene to cis- and trans-carveol and carvone, albeit at a relatively low rate

In a number of studies, strains with the capability to use D-limonene as the sole source of carbon and energy were isolated and subsequently assessed for the accumulation of degradation pathway intermediates during growth. Most of these studies were inconclusive as mixtures of oxidation products were detected and it generally remained unclear whether these were genuine pathway intermediates or dead-end products resulting from an incomplete regiospecificity of the pathway enzymes. A recent, more successful, example is the work of Van der Werf and De Bont who proved that Rhodococcus erythropolis DCL14 degraded D-limonene via epoxidation at the 1,2 position and managed to clone the gene encoding the epoxidase in Escherichia coli, resulting in a biocatalyst for the high-yield production of D-limonene-1,2-epoxide.

U.S. Pat. No. 5,688,673 discloses a process for the production of a mixture of carveol, carvone, perillyl alcohol, and terpineol using a DNA segment of a Bacillus stearothermophilus.

We have now discovered a range of strains expressing oxygenases which are capable of producing trans-carveol from D-limonene.

According to the present invention, there is provided a process for the production of trans-carveol which comprises (a) incubating in the presence of D-limonene, a culture medium comprising microbial cells expressing an oxygenase which is capable of dioxygenation of toluene and/or napththalene, and (b) recovering the carveol from the culture medium.

The process of the present invention is the only known method to use bacteria that is uniquely suitable to provide not only pure trans-carveol, but also this product in significant yields from D-limonene. Furthermore, the process is relatively simple and economical, thus making it attractive for use in an industrial application.

The process of the present invention relates to a process for the production of trans carveol from D-limonene using microbial strains expressing oxygenases which are capable of dioxygenation of toluene and/or napththalene. The microbial strain may suitably be bacterial, fungal or yeast. Preferably, the strain is bacterial. Strains capable of dioxygenation of toluene and/or napththalene are defined as strains being capable of degrading toluene through a pathway which involves the dioxygenation of the aromatic ring as an initial degradation step, generally referred to as the ‘tod’ pathway.

The process comprises providing a cell culture comprising the cells expressing such oxygenases and incubating the cells in the presence of D-limonene to produce isolatable amounts of trans-carveol. Strains suitable for use in the present invention include strains from the genus Rhodococcus, for example Rhodococcus opacus and Rhodococcus globerulus, and from the genus Pseudomonas. The preferred strain is that from the genus Rhodococcus and in particular, a strain Rhodococcus opacus PWD4 (DSMZ 44313).

The gene encoding the oxygenases may suitable be cloned and expressed in a bacterial or yeast strain.

The strain expressing the oxygenase must be capable of accepting toluene and/or naphthalene, preferably be capable of dioxygenation of toluene and/or naphthalene to the corresponding dihydrodiols, toluene-cis-1,2-dihydrodiol and cis-1,2-dihydroxynaphthalene.

The incubation may suitably be carried out at a temperature of from 15 to 40° C., preferably from 20 to 30° C.

The D-limonene is preferably provided in the culture medium in the gas phase or as a second phase which is liquid, either pure or dissolved in an inert solvent such as hexadecane. Suitably, the D-limonene is added in small increments over time directly to the culture medium. Such procedures are well known in the art.

The growth medium used in the cell culture may be any suitable mineral media including a suitable carbon source in the presence of a suitable inducer such as toluene.

The trans-carveol product may be isolated from the culture medium by solid phase extraction or by extraction with a suitable organic solvent. When D-limonene is used in the pure form, the product may be continuously extracted in the D-limonene phase. The D-limonene that is not converted may be removed by distillation under low pressure.

It has also been found that the trans-carveol product of the present invention may be partially or fully converted to carvone when a specific strain of enzyme is used. Thus, according to another aspect of the present invention there is provided a process for the production of trans-carveol and/or carvone which process comprises (a) incubating in the presence of D-limonene, a culture medium comprising Rhodococcus globerulus PWD8 (DSMZ 44306) and (b) recovering the carveol and/or carvone from the culture medium.

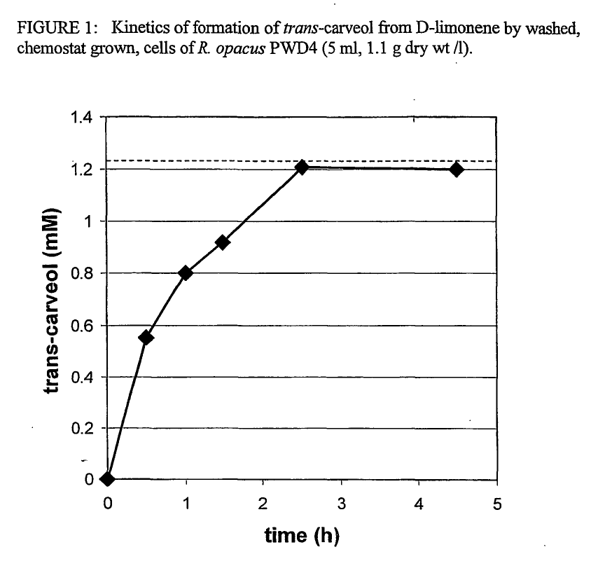

The present invention will now be illustrated with reference to the following examples and with reference to the accompanying FIG. 1:

FIG. 1 is a graph of the kinetics of formation of trans-carveol from D-limonene in the presence of Rhodococcus opacus PWD4.

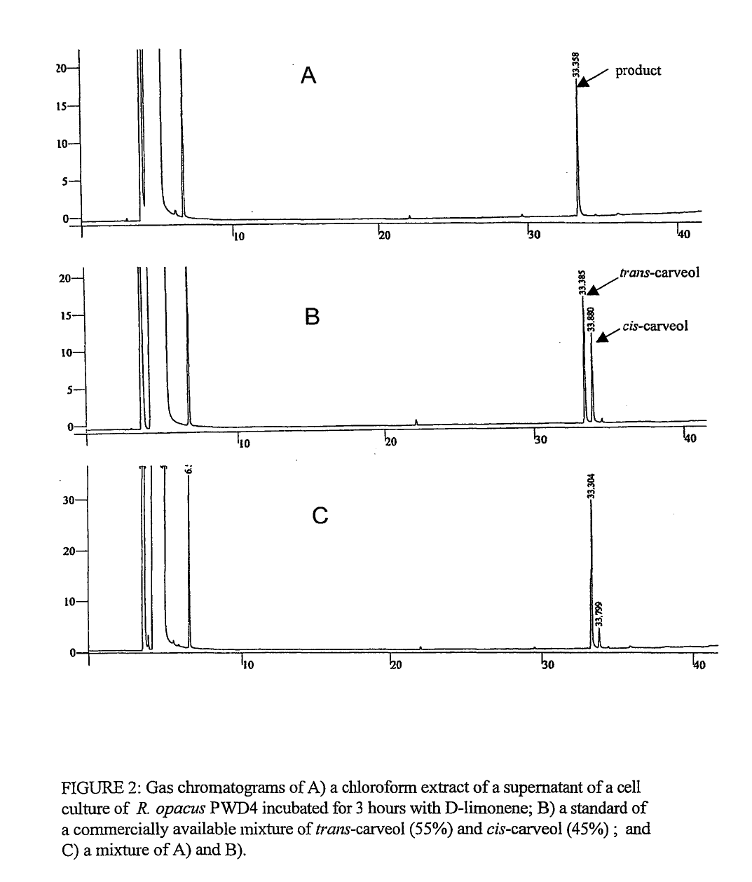

FIG. 2 is a chromatogram from the HPLC analysis of carveol.

### MATERIALS AND METHODS

(1) Screening: Multiple strains stored in one individual 96 well microtiter plate at minus 80° C. were sampled simultaneously (without thawing the remaining culture) using a spring-loaded 96-pin replicator (Kuhner, Basel, Switzerland) as previously described and transferred to a regular sterile polystyrene microtiter plate each well of which (working volume 350 μl) was filled with 180 μl of a solidified agar (2% [w/v]) mineral medium without any C-source. The inoculated microtiter plate (lid on top was kept at a distance of 2 mm from the wells, in order to allow for a sufficient level of gas diffusion in and out the wells) was placed in a dessicator together with a beaker of water and a 50 ml beaker containing 10 ml of a 10% solution (v/v) of toluene in hexadecane.

After 7 days of growth at ainbient temperature (22-25° C.) the cell mass developed on the agar surface was harvested as follows. 110 μl of a K₂HPO₄/KH₂PO₄-buffer (50 mM, pH 7.0) was added to each well. Repeated lateral movement of the spring-loaded replicator in the wells resulted in the suspension of a large part of the cell mass. The suspensions were subsequently transferred (using a 12 channel multipipette and wide orifice tips) to a microtiter plate with 0.5 ml conical wells (Maxi-plaque, Polylabo, Geneva). The microtiter plate was centrifuged for 15 min at 4000 rpm in a centrifuge (Eppendorf, type 5403). After disposal of the supernatant, the cells were resuspended in 100 μl of a K₂HPO₄/KH₂PO₄-buffer (50 mM, pH 7.0) by repeated filling and emptying of wide-orifice pipette tips (using a 12 channel multipipette). Subsequently, 2 μl of optically pure D-limonene (Fluka, p.a.) was added to each well and the wells were closed using a sandwich cover (a pierced layer of soft silicone in combination with a rigid polypropylene plate) as described previously (12), followed by 2 hours of orbital shaking at 300 rpm, 5 cm shaking amplitude, at 25° C. The microtiter plate was then centrifuged for 15 min at 4000 rpm. 50 μl of the supernatant (without the remaining D-limonene phase) was transferred into a half-area microtiter plate (Costar type 3696, Corning, N.Y.) using a 12 channel multipipette.

(2) Analytical Methods: The products were analysed by HPLC and GC. The HPLC (Agilent 1100 series) was equipped with a diode array detector (set at 190 nm) and a mass detector (Agilent) in series. The GC (Varian 3400) was equipped with a flame-inonisation detector (FID).

The results are given in Table 1 below. It can be seen from the results that the bacterial strains expressing oxygenases capable of dioxygenation of toluene are able to convert D-limonene to trans-carveol whilst the other strains are unable to convert D-limonene to trans-carveol.

TABLE 1
38 Toluene-degrading strains subdivided according to the concentration of trans-
carveol formed during a two-hour incubation with D-limonene. The strains that did not show any
detectable product formation are included in the lowest section. The last column indicates the
type of toluene biodegradation pathway harbored by the indicated strains.

| [Trans-carveol] | | | |
|---|---|---|---|
| (mM) | Taxonomy | strain codes | pathway |
| 500-1600 μM | Rhodococcus opacus | PWD4^{a)}, PWD7^{a)}, PWD10^{a,h)}, PWD19^{a,h)} | tod^{c)} |
| | Rhodococcus globerulus | PWD6^{a,g)}, PWD8^{a,g)}, PWD30^{a,h)} | tod^{c)} |
| | Rhodococcus erythropolis | PWD2^{a,d)}, PWD3^{a,d)} | tod^{c)} |
| | Pseudomonas syringae | PWD16^{b)} | tod^{c)} |
| | Pseudomonas sp. | PWD22 | tod^{c)} |
| | Alcaligenes sp. | PWD38^{h)} | tod^{c)} |
| 200-380 μM | Pseudomonas marginalis | PWD17^{b)}, PWD18^{b)}, PWD26^{b)}, PWD34^{b)} | tod^{c)} |
| | Pseudomonas syringae | PWD23^{b)}, PWD31^{b)} | tod^{c)} |
| | Rhodococcus globerulus | PWD1^{a)}, PWD28^{a,g)} | tod^{c)} |
| | Pseudomonas fluorescens | PWD37^{b)} | tod^{c)} |
| | Pseudomonas viridiflava | PWD12^{b)} | tod^{c)} |
| | Pseudomonas stutzeri | PWD32^{b)} | tod^{c)} |
| 50-180 μM | Alcaligenes sp. | PWD24^{b)} | tod^{c)} |
| | Pseudomonas syringae | PWD36^{b)} | tod^{c)} |
| | Rhodococcus opacus | PWD15^{a)} | tod^{c)} |
| below | Pseudomonas putida | GJ40, PWD33^{b)} | tod^{c)} |
| detection | Pseudomonas marginalis | PWD27^{b)} | tod^{c)} |
| limit | Pseudomonas putida | mt-2, | TOL^{d)} |
| | Pseudomonas syringae | PWD11^{b)}, PWD13^{b)} | TOL^{d)} |
| | Pseudomonas azotoformans | PWD20^{b)}, PWD39^{b)} | TOL^{d)} |
| | Pseudomonas stutzeri | PWD21^{b)}, PWD40^{b)} | TOL^{d)} |
| | Pseudomonas marginalis | PWD35^{b)} | TOL^{d)} |
| | Pseudomonas mendocina | KR-1 | KR1^{e)} |
| | Burkholderia cepacia | G4 | G4^{f)} |

^{a)}100% 16S RNA gene sequence identity with type strain
^{b)}99.0-99.8% 16S RNA gene sequence identity with type strain
^{c)}toluene degradation pathway involving dihydroxylation of the aromatic ring
^{d)}toluene degradation pathway involving monohydroxylation of the methyl group
^{e)}toluene degradation pathway involving monohydroxylation in the para-position (33)
^{f)}toluene degradation pathway involving monohydroxylation in the ortho-position (28)
^{g)}Co-producing carvone in concentrations of 8-16% of the trans-carveol concentrations
^{h)}Co-producing carvone in concentrations of 1-3% of the trans-carveol concentrations

### EXAMPLE 1

### Production of Trans-Carveol Using Rhodococcus opacus PWD4

1.1. Growth of Rhodococcus opacus PWD4:

Rhodococcus opacus was grown on a mineral medium supplied with 5 mM glucose in an 100 ml Erlenmeyer shaped chemostat at a dilution rate of 0.03 h⁻¹ . Toluene was supplied as an additional C-source via the gas phase as described previously, allowing the cells to reach a dry weight of approximately 1 g dry wt per liter.

1.2. Production of Trans-Carveol Using Rhodococcus opacus PWD4:

Rhodococcus opacus PWD4 was cultivated in chemostat culture at a dilution rate of 0.03 h⁻¹ on a mineral medium with toluene, supplied via the gas-phase, as the main C-source. After the culture had reached a constant dry weight of 1.25 g dry wt 1⁻¹, cells were harvested and used to study the biotransformation kinetics of D-limonene in the presence and absence of toluene In the absence of toluene, the maximal concentration of trans-carveol was measured after 2 hours (1.21 mM) (FIG. 1). The only other oxidation product detected was carvone (˜15 μM, or 1.3% of the trans-carveol formed). The molar yield of trans-carveol in the experiment without toluene was approximately 97% (mol/mol) if the standard of carveol is assumed to be 100% pure, or 94% (mol/mol) if the standard of carveol is only 97% (indicated as the minimum purity by the supplier). A gas chromatogram of a chloroform extract of this biotransformation showed only one significant peak, corresponding exactly to the retention time of trans-carveol (FIG. 1). The initial specific activity was 14.7 U (g cell dry wt)⁻¹.

The experiment was done in a closed headspace vial with a total volume of 43 ml. The dashed line indicates the theoretical concentration of trans-carveol if the molar yield on D-limonene were 100%

In the presence of toluene, the specific acitivity of trans-carveol formation was 0.8 U (g cell dry wt)⁻¹. The average aqueous toluene depletion rate in the first hour of incubation was 22 μM min⁻¹, corresponding to an specific acitivity of toluene consumption (after correction for the toluene [68% of total amount] present in the gaseous form in the headspace) of 58 U (g cell dry wt)⁻¹. After all toluene was depleted, the specific trans-carveol formation rate increased 9-fold to 7.8 U (g cell dry wt)⁻¹. Comparison of the maximal toluene degradation rates with the maximal trans-carveol formation rates measured indicates that the relative activity on D-limonene was 25%. These results show that toluene is a reversible competitive inhibitor of the biotransformation of D-limonene to trans-carveol.

### EXAMPLE 2

### Co-Production of Trans Carveol and Carvone Using Rhodococcus globerulus PWD8

Toluene-induced cells of the strain, Rhodococcus globerulus PWD8, were incubated with D-limonene. The initial trans-carveol concentration (average of the first hour) was 0.73 mM h⁻¹ corresponding to a specific activity of 3 U (g dry wt)⁻¹. After two hours, more than 90% of D-limonene added was transformed to trans-carveol (1.1 mM) and a concentration of 0.08 mM carvone was formed. The concentration of carvone gradually increased to maximally 0.29 mM after 27 hrs, while the trans-carveol concentration decreased accordingly, indicating that the trans-carveol formed was converted slowly to carvone.

1. A process for the production of trans-carveol which process comprises incubating in the presence of D-limonene, a medium comprising microbial cells expressing an oxygenase capable of dioxygenation of toluene and/or napththalene and optionally recovering the trans-carveol from the culture medium.

2. A process as claimed in claim 1 in which the microbial cells are fungal, bacterial or yeast cells.

3. A process as claimed in claim 2 in which the cells are bacterial.

4. A process as claimed in claim 1 in which the cells expressing the oxygenase are strains from the genus Rhodococcus or the genus Pseudomonas.

5. A process as claimed in claim 4 in which the strain belongs to the genus Rhodococcus.

6. A process as claimed in claim 5 in which the Rhodococcus is Rhodococcus opacus PWD4 (DSMZ 44313).

7. A process as claimed in claim 1 in which the microbial cells are recombinant bacterial or yeast cells and the oxygenase expressed therein is a heterologously expressed oxygenase.

8. A process as claim in claim 1 in which the D-limonene is present in the culture medium in the gas phase or in the liquid phase.

9. A process as claimed in claim 1 in which the incubation is carried out at a temperature of from 15 to 40° C.

10. A process for the production of trans-carveol and/or carvone which process comprises (a) incubating in the presence of limonene, a culture medium comprising Rhodococcus globerulus PWD8 (DSMZ 44306) and (b) recovering the carveol and/or carvone from the culture medium.

11. A process as claimed in claim 10 in which the trans-carveol formed is converted to carvone.

12. A process as claimed in claim 1 in which the incubation is carried out at a temperature of from 20 to 30° C.
